(19) United States
(12) Patent Application Publication
(10) Pub. No.: US 2023/0059785 A1
(43) Pub. Date: Feb. 23, 2023

(54) METHODS FOR ENHANCING T CELLS USING VENETOCLAX

(71) Applicant: University Health Network, Toronto (CA)

(72) Inventors: Li Zhang, Toronto (CA); Jongbok Lee, Toronto (CA); Aaron David Schimmer, Thornhill (CA)

(21) Appl. No.: 17/797,630

(22) PCT Filed: Feb. 8, 2021
(86) PCT No.: PCT/CA2021/050138

§ 371 Date: Aug. 4, 2022

Related U.S. Application Data
(60) Provisional application No. 62/971,534, filed on Feb. 7, 2020.

(51) Int. Cl. A61K 35/17; A61K 31/635; A61P 35/00

(52) U.S. Cl. CPC A61K 35/17; A61K 31/635; A61P 35/00

(57) ABSTRACT

Methods of treating T cells with Venetoclax to increase T cell-mediated cytotoxicity and/or T cell mediated anti-tumor activity are described. Also described are populations of enhanced T cells as well as associated methods and uses for the treatment of cancer.

### RELATED APPLICATIONS

This application claims priority to U.S. provisional application No. 62/971,534 filed Feb. 7, 2020, the entire contents of which are hereby incorporated by reference.

### FIELD

The disclosure relates to immunotherapy for the treatment of cancer and more specifically to enhancing T cells for the treatment of cancer using Venetoclax.

### BACKGROUND OF THE INVENTION

Adoptive cellular therapy (ACT) has significantly improved outcomes of patients with certain cancer types such as B cell leukemia and melanoma (1, 2). While these successes demonstrate the potency of ACT, similar clinical benefits have not been obtained for other cancer types. For example, ACT for acute myeloid leukemia (AML), which presents highly heterogenous disease both within and amongst patients, has not been clinically successful despite various approaches of ACT being investigated in attempt to improve the outcomes of patients otherwise suffering from this highly lethal disease (3). Therefore, there remains a need for improved ACT therapies for the treatment of cancer.

One form of ACT uses a unique subset of T cells defined as CD4- and CD8-double negative T (DNT) cells. In preclinical models, unlike many other T cell therapies, infusion of allogeneic DNT cells expanded from healthy volunteers does not induce alloreactivity against normal cells and are resistant to immune rejection by recipients, collectively supporting their potential to be used as an off-the-shelf ACT (3-6). However, the anti-cancer effect of DNT cells is not complete (5, 6), hence approaches that can further enhance DNT cell anti-tumor activity may lead to a better patient outcome.

### SUMMARY

In one aspect, it has been determined that the Venetoclax enhances T cell treatment efficacy by increasing T cell-mediated cytotoxicity.

T cells were pretreated with compounds from a library of 269 drugs approved for various clinical uses and, subsequently, compound treated cells were used as effectors against a human AML cell line. Surprisingly, the Bcl-2 inhibitor Venetoclax increased the cytotoxicity of T cells the most. (FIG. 1).

As set out in the Examples, T cells pre-treated with Venetoclax showed enhanced T cell-mediated cytotoxicity against AML in vitro. Moreover,

Venetoclax-treated T cells showed increased anti-tumoral activity in a xenograft model. Venetoclax, but not other Bcl-2 family protein inhibitors, enhanced the cytotoxicity of T cells. Compared to untreated T cells, Venetoclax-treated T cells had higher expression of the T cell activation markers CD25 and CD69, and higher expression of effector molecules NKG2D and DNAM-1. Venetoclax-treated T cells also showed increased levels of reactive oxygen species (ROS) compared to untreated cells. Therapeutically relevant concentrations of Venetoclax were also demonstrated to increase T cell effector function without decreasing T cell viability. Furthermore, T cells isolated from patients receiving Venetoclax demonstrated increased levels of ROS.

Accordingly, in one embodiment there is provided a method of enhancing the therapeutic efficacy of T cells, comprising contacting T cells with Venetoclax to produce functionally enhanced T cells.

The use of Venetoclax for pre-treatment of T cells as described herein produces enhanced T cells that have a number of characteristics that make the cells more effective for the treatment of cancer. For example, in one embodiment, the use of Venetoclax increases T cell-mediated cytotoxicity. In one embodiment, the use of Venetoclax increases T cell-mediated anti-tumor activity. In one embodiment, contacting the T cells with Venetoclax increases the relative proportion of T cells in an effector memory state.

In one embodiment, the T cells are conventional T cells (CD4⁺ or CD8⁺). In one embodiment, the T cells are non-conventional T cells such as double negative T cells (CD4⁻, CD8⁻).

In one embodiment, the method comprises contacting the T cells with a concentration of Venetoclax of at least 50 nM. In one embodiment, the method comprises contacting the T cells with a concentration of Venetoclax of at least 100 nM, at least 200 nM, at least 300 nM or at least 400 nM, optionally a concentration of Venetoclax between about 100 nM and about 1 μM.

In one embodiment, the method comprises contacting the T cells with Venetoclax for at least about 30 minutes, at least about 45 minutes or at least about 60 minutes. In one embodiment, the method comprises contacting the T cells with Venetoclax for at least 1 hour, at least 1.5 hours, at 2 hours or at least 4 hours. In one embodiment, the method comprises contacting the T cells with Venetoclax for at least 6 hours, at least 8 hours or at least 12 hours, optionally between about 1 hour and about 7 days. In one embodiment, the method comprises contacting the T cells with Venetoclax for at least 1 hour and less than about 14 days, 10 days, 9 days, 8 days, 7 days, 6 days or 5 days. In one embodiment, the method comprises contacting the T cells with Venetoclax for a period of time sufficient to increase the level of expression of one or more of CD25, CD69, NKG2D, DNAM-1, and NRF2 by the T cells relative to control cells not contacted with Venetoclax. In one embodiment, the method comprises contacting the T cells with Venetoclax for a period of time sufficient to increase the level of cellular reactive oxygen species (ROS) relative to control cells not contacted with Venetoclax. In one embodiment, the T cells are in vitro. In another embodiment, the T cells are in vivo or ex vivo.

The enhanced T cells described herein are readily distinguished from T cells that have not been pre-treated with Venetoclax. In one embodiment, contacting the T cells with Venetoclax increases the level of expression of one or more of CD25, CD69, NKG2D, DNAM-1, and NRF2. In one embodiment, contacting the T cells with Venetoclax increases the level of cellular reactive oxygen species (ROS).

Also provided is a population of enhanced T cells produced by a method described herein. In one embodiment, the enhanced T cells exhibit an increased level of expression of one or more of CD25, CD69, NKG2D, DNAM-1, and NRF2 relative to control T cells not contacted with Venetoclax. In one embodiment, the enhanced T cells exhibit an increased level of cellular reactive oxygen species (ROS) relative to control T cells not contacted with Venetoclax.

In one embodiment, the proportion of T cells in an effector memory state relative to T cells in a naïve state in the population of enhanced T cells is increased compared to the proportion of T cells in an effector memory state relative to T cells in a naïve state in a control population of T cells not contacted with Venetoclax.

In one embodiment, there is provided a composition comprising T cells and Venetoclax. Also provided is a pharmaceutical composition comprising enhanced T cells treated with Venetoclax as described herein.

Also provided is the use of the enhanced T cells, compositions and/or a combination of T cells and Venetoclax as described herein for the treatment of cancer in a subject in need thereof. In one embodiment, there is provided a method of treating cancer in a subject in need thereof, the method comprising administering to the subject enhanced T cells, compositions and/or a combination of T cells and Venetoclax as described herein. In one embodiment, the cancer is leukemia, optionally acute myeloid leukemia (AML).

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

One or more embodiments of the disclosure will now be described in relation to the drawings in which:

FIG. 1. Drug screening assay identifies Venetoclax as the top hit for enhancing cytotoxicity of T cells against AML. Schematic diagram of drug screening assay done to identify clinically approved drugs that can be used in combination with DNT cells to yield in synergistic anti-tumor activity. DNT cells were treated with 269 different clinically approved drugs at 400 nM for overnight. Subsequently, compound-treated cells were washed and then cultured with AML cells for two-hours. Dot plot shows the changes in the degree of cytotoxicity mediated by DNT cells against AML cells relative to the untreated DNT cells.

FIG. 2. Venetoclax enhances T cell mediated cytotoxicity against AML in vitro. (A) To validate the finding from the drug screening, in vitro killing assay was conducted with DNT cells untreated or pretreated with various concentrations of Ven (50 nM, 100 nM, 200 nM, 400 nM) for overnight against AML cell lines, OCI-AML2, OCI-AML3, and KG1a. The data is representative of four biological replicates. (B) In vitro cytotoxicity assay using DNT cells pretreated with 400 nM Ven as effectors against primary AML patient samples (n=17). (C) To determine the activity of DNT cells with or without Ven-treatment against leukemia initiating cells, untreated AML or AML treated with untreated or Ven-treated DNT cells were seeded at 10³ cells per ml in a methylcellulose-based colony forming assay, and the number of colonies formed were determined 10 days after. The experiment was done with OCI-AML2 and KG1a, as well as patient samples 140372, 100857, 110162 and 141065. (D) The increased effector activity by Venetoclax treatment was retained for at least four days after the removal of the drug from the DNT cells against three AML cell lines, OCI-AML2, OCI-AML3, and KG1a. The experiment was done using DNT cells from two different donors (UPN119 and UPN38). (E) Correlation between the susceptibility of AML to DNT cells and the degree of increase in DNT cell-mediated cytotoxicity by Ven treatment. (F) DNTs expanded from 11 donors were untreated or treated with 400 nM Venetoclax for 18 hours. Subsequently, they were cultured with OCI-AML2 at 1:1, 2:1, or 4:1 DNT:AML ratio, and the viability of AML cells were measured by Annexin V staining and flow cytometry. Each paired symbol represents DNTs from an individual donor.

FIG. 3. Pre-treating DNT cells with Ven increase their anti-tumoral activity in a xenograft model. To determine if Ven pretreated DNT cells induce greater anti-leukemic activity in a xenograft model, NOD/SCID mice subcutaneously engrafted with 2×10⁶ OCI-AML2 cells were intravenously infused with PBS (●), 2×10⁷ untreated DNT cells (▪), or 2×10⁷ Ven-treated DNT cells (▴) when tumor size reached 100 mm³ (indicated by an arrow). Tumor volume was monitored until the PBS-treated group reached a humane endpoint (A) and tumor weight was measured on day 20 after leukemia inoculation (B). The results shown are representative of three independent experiment done using DNT cells from three different donors. (C) NSG mice systemically infused with KG1a were treated with PBS, DNT cells, or VenDNT cells. Bone marrow engraftment of KG1a were compared between the groups. VenDNT treated mice show significantly lower levels of KG1a engraftment compared to PBS and DNT cell treated groups, further supporting the superior anti-leukemic activity of VenDNT cells even against those otherwise resistant. (D) Primary AML cells (ID: 130607) untreated or treated with DNTs or Ven-treated DNTs for 2 hours at 2:1 DNT:AML ratio were injected intrafemorally into NOD/SCID mice (1.6×10⁶ cells per mouse; n=6 per group). Six weeks after injection, the percent of AML engraftment (human CD45⁺ CD33⁺ cells) in the bone marrow from each group was determined by flow cytometry. (E) Sublethally irradiated NSG mice were intravenously injected with primary AML cells (n=4; 2−5×10⁶/mouse). Two weeks later, mice were treated with three infusions of vehicle control or 1.5−2×10⁷ cells per infusion of DNTs or Ven-treated DNTs, 3-4 days apart. Five weeks post AML injection, bone marrow engraftment of primary AML cells (human CD45^{low} CD33⁺ with or without CD34 expression) was determined by flow cytometry. (Left) Representative contour plot of BM cells from each group stained with CD45 and CD33. (Right) Summarized results from patient-derived xenograft experiments performed using four different primary AML patient samples. Horizontal bar represents the mean of BM AML engraftment level normalized to vehicle control group, each symbol represents individual mouse, and error bars represent SD. Data represent the mean ±SEM reduction in bone marrow leukemia level relative to PBS group. Student's t-test or one-way ANOVA were used for statistics. *p<0.05, **p<0.01, ***p<0.001, ****p<0.0001.

FIG. 4(A). Venetoclax enhances anti-leukemic activity of CD4⁺ or CD8⁺ conventional T cells. Ex vivo expanded T_{conv} cells untreated or treated with various concentrations of Ven (25 nM, 50 nM, 100 nM, 200 nM, or 400 nM), were used as effector cells against AML cell lines, OCI-AML2, OCI-AML3, and KG1a. The result shown represents four biological replicates. FIG. 4(B).

Venetoclax rapidly and directly increases cytotoxicity of T cells against AML. DNT (top panels) and T_{conv} cells (bottom panels) untreated or treated with Venetoclax (100 nM and 400 nM) for 4 h, 18 h, and 3 days. Subsequently, their cytotoxicity against OCI-AML2 was determined. Data represent the mean ±SEM of results from four different donor T cells. FIG. 4(C). DNT and T_{conv} cells untreated or treated with Venetoclax (100 nM or 400 nM) for 4 hours. Subsequently, their viability was determined. Data represent the mean ±SEM of results from four different donor T cells.

FIG. 5. Venetoclax, but not Obatoclax or ABT-737, enhances anti-leukemic activity of DNT cells. (A) DNT cells were pre-treated with different concentrations of Obatoclax, ABT-737, or Venetoclax overnight and were used as effector cells against OCI-AML2. (B) The results show the percentage change in DNT-mediated cytotoxicity compared to the degree of killing induced by untreated DNTs. (C) Expression of Bcl-xL and Bcl-2 on ex vivo expanded DNT cells from three donors (UPN38, UPN108, and UPN134) and AML cell lines, OCI-AML2, TEX, NB4, and K562 determined by Western blot. Tubulin was used as a loading control.

FIG. 6. Ven increases expression of activation markers and effector molecule on DNT cells. Ex vivo expanded DNT cells were untreated or treated with 400 nM Ven and were stained for expression of T cell (A) activation markers CD25 and CD69, and (B) effector molecules (NKG2D and DNAM-1. Each pair of dots represents DNT cells derived from one donor before and after Ven treatment. The experiment was done using DNT cells from four (A) or six (B) different donors. (C) Expression of granzyme B in DNT cells treated with different concentrations of Ven. The result shown represents two biological replicates. (D) A dose-dependent increase in CD25, NKG2D, and DNAM-1 expression was also observed on Ven treated CD8⁺ T cells.

FIG. 7. Ven increases cellular ROS level in DNT cells and enhances their cytotoxic activity. (A) level of cellular ROS in DNT cells (Left) or CD8+ T cells (right) treated with different concentrations of Ven detected by CellROX™ staining. (B) (Left) Relative expression of a transcription factor regulated by cellular ROS level, Nrf2, determined by qPCR. (Right) Nrf2 Western blot in cytoplasmic and nuclear faction of DNTs with or without 400 nM Ven treatment to determine location of Nrf2 protein. The data was generated using DNTs from three different donors (UPN38, UPN108, and UPN134). (C) To determine the functional relevance of increased ROS level in Ven treated DNTs, ROS level in DNTs treated with 400 nM Ven in the presence of various concentrations of ROS-scavenger, N-acetylcysteine (NAC), and these cells were used as effector cells against AML during an in vitro killing assay. The result shown is representative of three independent experiments. (D) To determine the source ROS production in Ven-treated DNTs, native gel and immunoblotting was done on DNTs untreated or treated with 400 nM for detection of components of electron transport chain supercomplex subunits (NDUFA9, UQCRC2, and MTCO1). The results shown is representative of three independent experiments done with DNTs derived from two different donors. (e and f) Ven increased the proportion of cells in effector memory stage while reducing the frequency of central memory T cells for both DNT cells (E) and CD8⁺ T_{conv} cells (F). (G) Ven had no significant effect on glycolysis, glycolytic capacity, and basal oxygen consumption rate of DNT cells. (H-K) DNT (H and I) or T_{conv} cells (J and K) were treated with 0 nM, 100 nM, or 400 nM Venetoclax for 4 hours, 18 hours and 2 days. Cells were stained with CellROX (H and J) or MitoSOX (I and K). MFI of cellular or mitochondrial (mt) ROS was measured by flow cytometry. Data represent the mean ±SEM of results from four different donor T cells. (L) DNTs treated with 400 nM Venetoclax with or without 2 mM NAC for 18 hours. Flow histogram shows the cellular ROS level measured by flow cytometry. MFI of CD25 and CD69 were measured by flow cytometry. Experiments were done in triplicates, and the data shown is representative of two independent experiments done using DNTs from two donors. (M) DNT cells were treated with 400 nM Venetoclax for 18 hours. After treatment, mitochondria were isolated and levels of respiratory chain complex subunits were measured by SDS-PAGE gels and immunoblotting with antibodies against NDUFB8 (complex I), SDHA (complex II), UQCRC2 (complex III), MTCO1 (complex IV).

FIG. 8. Patients treated with Ven+Aza have increased proportion of T cell subsets associated with cytotoxic activity. Patient peripheral blood samples were obtained before and on 4 day of Ven+Aza treatment, and the frequency of different T cell subsets, effector molecule expression, and cellular ROS level was determined by flow cytometry. (A) The frequency of CD8⁺ and DNT cells were compared between samples obtained before and after Ven+Aza treatment. (B-E) Frequency of effector memory T cell subset (CD45RA⁻ CD62L⁻), and expression level of NKG2D and cellular ROS level were compared within CD8⁺ T (b and c) and DNT (D and E) cell populations. The graphs shown are summary of results of samples taken from four patients

FIG. 9. Insignificant degree of killing was seen with both Ven-treated and -untreated DNT cells against autologous and allogeneic PBMCs.

FIG. 10. Venetoclax does not kill DNTs while enhancing their cytotoxicity against AML. (A) Viability of DNTs and OCI-AML2 cells treated with 400 nM Venetoclax for 18 hours was determined by Annexin V staining and flow cytometry. (B and C) DNTs were treated with increasing concentrations of Venetoclax for 18 h. Subsequently, their viability (B) and cytotoxicity (C) against OCI-AML2 and two primary AML cells (090765 and 110162) were determined. ANOVA was used for statistics. ****p<0.0001.

FIG. 11. Venetoclax has comparable effect on DNT-mediated cytotoxicity against diagnostic and relapsed/refractory AML samples. 400 nM Venetoclax treated or untreated DNTs were cocultured with diagnostic (n=12) or relapsed/refractory (n=4) primary AML samples at 2:1 ratio for 2 hours. The increase in DNT-mediated cytotoxicity by Venetoclax treatment was determined against each patient sample type.

FIG. 12. DNTs to induce superior anti-leukemic activity in the presence of Venetoclax. (A) KG1a and OCI-AML2 cells were untreated or treated Venetoclax (100 nM) in the presence or absence of DNTs. (B) % reduction in AML counts by DNTs in the presence or absence of Venetoclax (100 nM).

FIG. 13. Ven-treated DNTs induce greater reduction in total AML number without increasing T cell engraftment in bone marrow. Sublethally irradiated (250cGy) NSG mice were injected intravenously with KG1a cells (2×10⁶ cells/mouse) or primary AML cells. Two weeks later, mice were treated with three infusions of vehicle control (PBS) or 1.5−2×10⁷ cells per infusion of DNTs or Ven-treated DNTs 3-4 days apart. Five weeks post AML injection, AML cell counts (A) and the frequency of T cells (B) in the bone marrow were determined by staining bone marrow cells with anti-human CD45, CD3, CD33, and CD34 antibodies and flow cytometry analysis.

FIG. 14. Untreated and Venetoclax treated DNTs do not cause tissue damage. Sublethally irradiated (250cGy) NSG mice were injected intravenously with KG1a cells (2×10⁶ cells/mouse). Two weeks later, mice were treated with three infusions of vehicle control (PBS) or 1.5−2×10⁷ cells per infusion of DNTs Ven-treated DNTs 3-4 days apart. On day 35, liver (top) and lung (bottom) tissues were stained with hematoxylin and eosin (H&E) (50× magnification). PV—portal vein; ALV—alveoli; BR—bronchioles.

FIG. 15. Effect of other known ROS-inducing reagents on DNT viability, ROS level, and cytotoxicity against AML. DNTs were treated with increasing concentrations of cytarabine (0-3 μM), antimycin (0-250 nM) or daunorubicin (0-10 μM) for 18 hours. Subsequently, the level of cellar ROS in the DNTs (A), DNT viability (B), and cytotoxicity against OCI-AML2 (C) were determined.

FIG. 16. Venetoclax does not affect the expression of electron transport chain (ETC) complex subunits. The relative levels of the proteins were normalized to loading control MnSOD and were expressed as relative to the value of control which was set to 1.0. Representative immunoblots are shown. Data are represented as mean ±SD from three independent experiments.

### DETAILED DESCRIPTION

The pre-treatment of T cells with Venetoclax has been shown to increase T-cell mediated cytotoxicity and anti-tumor activity both in vitro and in vivo. T cells contacted with Venetoclax and associated compositions as well as combinations of T cells and Venetoclax are therefore expected to be useful for the treatment of subjects with cancer.

### I. Methods of Enhancing T Cells and Populations Thereof

In one embodiment, there is provided a method of enhancing the therapeutic efficacy of T cells comprising contacting the T cells with Venetoclax to produce enhanced T cells.

The term “Venetoclax” or “Ven” as used herein refers to a molecule capable of binding to and inhibiting Bcl-2. In one embodiment, Venetoclax is the drug Venclexta™ or the drug Venclyxto™.

In one embodiment, the method further comprises contacting cancer cells with Azacytidine or the administration or use of Azacytidine in combination with enhanced T cells as described herein. The term “Azacytidine” or “Azacitidine” or “5-azacytidine” as used herein refers to compound that is a pyrimidine nucleoside analog of cytidine having antineoplastic activity. Proper chemical names of azacytidine include 4-amino-1-β-D-ribofuranosyl-1,3,5-triazin-2(1H)-one or 4-amino-1-[3,4-dihydroxy-5-(hydroxymethyl)oxolan-2-yl]-1,3,5-triazin-2-one.

The term “T cell” as used herein includes thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. T cells may be obtained by a person of skill in the art. T cells can by either conventional T cells (Tconv) or non-conventional T cells such as double negative T cells (DNTs) gamma-delta T cells or NKT cells. In one embodiment, the T cells are activated T cells. In one embodiment, the T cells are cells that have been expanded and/or activated ex vivo or in vitro.

T cells can readily be obtained and/or isolated from e.g. biological sources such as a blood sample or cell culture. For therapeutic applications, the T cells may be autologous T cells or allogenic T cells. In one embodiment, the T cells are autologous T cells obtained from a subject, such as a subject with cancer or suspected of having cancer. In another embodiment, the T cells are allogenic, such as T cells obtained from one or more subjects without cancer. In one embodiment, the T cells are obtained from one or more healthy donors.

DNTs can be obtained by enriching using CD4 and CD8-depetion antibody cocktails. In one embodiment, the DNTs do not express CD4 and CD8. In one embodiment, the DNTs have the phenotype CD3+, γδ-TCR+ or αβ-TcR+, CD4-, CD8-, α-Gal-, CTLA4-. In one embodiment, the DNTs have the phenotype CD3+, γδ-TCR+ or αβ-TcR+. In one embodiment, the DNTs may be obtained from a sample comprising peripheral blood mononuclear cells (PBMC). In one embodiment, the sample is a blood sample. In one embodiment, the sample is an apheresis sample, or an enriched leukapheresis product such as a leukopak. In one embodiment, the sample is a bone marrow sample.

In one embodiment, the T cells are expanded in vitro or ex vivo before being contacted with Venetoclax. Exemplary methods for isolating and expanding DNTs are described in U.S. Pat. No. 6,953,576 “Method of Modulating Tumor Immunity”, PCT Publication No. WO2007/056854 “Method of Expanding Double Negative T Cells”, and PCT Publication No. WO2016/023134 “Immunotherapy for the Treatment of Cancer” all of which are hereby incorporated by reference in their entirety.

The term “enhanced T cells” or “enhanced T cell” as used herein refers to individual T cells or a population of T cells that exhibit increased cytotoxic and/or anti-tumor activity following contact with Venetoclax compared to control T cells that have not been contacted with Venetoclax. Optionally, the enhanced T cells may be DNTs or conventional T cells (Tconv). In one embodiment, enhanced T cells may be distinguished from other T cells and/or control T cells on the basis of physiological activity and/or gene expression. For example, in one embodiment enhanced T cells exhibit an increased level of expression of one or more of CD25, CD69, NKG2D, DNAM-1, and NRF2 relative to control T cells not contacted with Venetoclax. In one embodiment enhanced T cells exhibit an increased level of expression of 2, 3, 4 or 5 genes selected from CD25, CD69, NKG2D, DNAM-1, and NRF2 relative to control T cells not contacted with Venetoclax

The term “contacted” or “contacting” as used herein refers to any method of exposing T cells to Venetoclax to produce enhanced T cells. “Contacting” includes “incubating” and “exposing” and does not imply any specific time or temperature requirements, unless otherwise indicated. In one embodiment, the T cells are contacted with Venetoclax in vitro, such as by combining Venetoclax with a culture media and exposing or incubating the T cells in the culture media. T cells may be “contacted” with Venetoclax via incubation in vitro, or by administration or co-administration to a subject such that the T cells are “contacted” with Venetoclax in vivo.

In one embodiment, the T cells are contacted with Venetoclax in vitro, ex vivo or in vivo at a concentration of at least 25 nM, 50 nM or 100 nM. In one embodiment, the T cells are contacted with a concentration of Venetoclax of at least 100 nM, at least 200 nM, at least 300 nM or at least 400 nM. In one embodiment, the T cells are contacted with a concentration of Venetoclax between about 10 nM and 10 μM, optionally between about 50 nM and 500 nM, between about 50 nM and 800 nM, or between about 100 nM and about 1 μM.

In another embodiment, the T cells are contacted with Venetoclax for at least about 30 minutes, 45 minutes, 60 minutes or 90 minutes. In one embodiment, the T cells are contacted with Venetoclax for at least about 1 hour, 2 hours, 4 hours, 6 hours, 12 hours, 18 hours, 24 hours, 36 hours or 48 hours. In one embodiment, the T cells are contacted with Venetoclax for between about 1 hour and 14 days, optionally 2 hours and 30 days, between about 4 hours and 14 days, between about 4 hours and 6 days, between about 4 hours and 48 hours, or between about 6 hours and about 24 hours. In one embodiment, the T cells are contacted with Venetoclax for less than about than about 14 days, 10 days, 9 days, 8 days, 7 days, 6 days or 5 days.

In one embodiment, the T cells are contacted with a sufficient concentration of Venetoclax for a sufficient time to increase the expression of one or more of CD25, CD69, NKG2D, DNAM-1, and NRF2. In one embodiment, the T cells are contacted with a sufficient concentration of Venetoclax for a sufficient time to increase the level of cellular ROS.

In some embodiments, after the T cells are contacted with the Venetoclax to become enhanced T cells, some or all of the Venetoclax may be removed or the enhanced T cells are isolated to reduce the concentration or extra-cellular Venetoclax.

Contacting T cells with Venetoclax as described herein produces an enhanced T cell population exhibiting a number of characteristics that render them particularly useful for the treatment of cancer. For example, in one embodiment Venetoclax increases T cell-mediated anti-tumor activity. In one embodiment, the Venetoclax increases T cell-mediated cytotoxicity.

The term “anti-tumor activity” as used herein refers to any activity of killing tumor cells and/or inhibiting tumor growth. In one embodiment, “anti-tumor activity” comprises reducing colony formation of tumor cells.

The term “cytotoxicity” as used herein refers to the quality of effecting cell death, causing cells to become cytostatic, and/or preventing cells from proliferating.

### II. Products, Compositions and Kits

In another aspect, there is provided a population of enhanced T cells produced according to the methods described herein. Also provided are compositions comprising enhanced T cells as described herein. For example, in one embodiment, the enhanced T cells are in a pharmaceutical composition, optionally with a pharmaceutically acceptable carrier.

In another embodiment there is provided a composition comprising T cells and Venetoclax. In one embodiment, the composition further comprises a cell culture media.

Also provided is a kit comprising T cells and Venetoclax. In one embodiment, the kit further comprises instructions for performing a method described herein, such as for producing enhanced T cells, for the treatment of cancer or for reducing the growth or proliferation of a tumor. In one embodiment, the T cells and the Venetoclax are in separate containers. In one embodiment, the T cells and the Venetoclax are in the same container, optionally as a composition with a pharmaceutically acceptable carrier.

Also provided is the use of the products, compositions or kits described herein for use in the treatment of cancer or in the preparation of a medicament for the treatment of cancer.

### III. Methods and Uses of Treating Cancer and Reducing the Growth and Proliferation of a Tumor

Enhanced T cells produced by the methods described herein have increased cytotoxicity against AML cells in vitro compared to T cells not treated with Venetoclax. As shown in Example 2, AML cells treated with enhanced T cells exhibited more specific killing of AML cells and less colony formation, compared to AML cells treated with control T cells. Moreover, Example 3 demonstrates that enhanced T cells have greater anti-tumoral activity in xenograft models.

Accordingly, in one embodiment there is provided a method of treating cancer in a subject in need thereof. In one embodiment, the method comprises administering to the subject an effective amount of enhanced T cells.

In one embodiment, the enhanced T cells are produced by contacting the T cells with Venetoclax as described herein. In one embodiment, the method comprises administering to the subject T cells and Venetoclax, optionally combined in a composition with a pharmaceutically acceptable carrier, wherein the T cells are enhanced by contact with Venetoclax in vivo.

Also provided is a method for reducing the growth and/or proliferation of a tumor. In one embodiment, the method comprises contacting the tumor with an effective amount of enhanced T cells. In one embodiment, the enhanced T cells are produced by contacting the T cells with Venetoclax as described herein.

Also provided is the use of enhanced T cells, compositions, and/or kits as described herein for the treatment of cancer in a subject in need thereof. In one embodiment, the enhanced T cells are produced according to a method described herein. In one embodiment, the enhanced T cells, compositions, and/or kits are for use in the manufacture of a medicament for the treatment of cancer. In one embodiment, the use comprises the use or administration of enhanced T cells to the subject. In another embodiment, the use comprises the use or administration of Venetoclax and T cells to a subject at the same time, or at different times.

Also provided are uses to reduce the growth and proliferation of a tumor. In one embodiment, the enhanced T cells, compositions, and/or kits described herein are for use in reducing the growth and proliferation of a tumor. In one embodiment, the enhanced T cells, compositions, and/or kits are for use in the manufacture of a medicament to reduce the growth and proliferation of a tumor. In one embodiment, the enhanced T cells and/or compositions are for use in the manufacture of a medicament to reduce the growth and proliferation of a tumor. In one embodiment, the T cells and Venetoclax are for use in the manufacture of a medicament to reduce the growth and proliferation of a tumor.

As used herein, the term “cancer” refers to one of a group of diseases caused by the uncontrolled, abnormal growth of cells that can spread to adjoining tissues or other parts of the body. In one embodiment, the cancer is a leukemia such as acute myeloid leukemia (AML).

The term “cancer cell” refers a cell characterized by uncontrolled, abnormal growth and the ability to invade another tissue or a cell derived from such a cell. Cancer cells include, for example, a primary cancer cell obtained from a patient with cancer or cell line derived from such a cell. In one embodiment, the cancer cell is a leukemia cell such as an AML cell.

The term “leukemia” as used herein refers to any disease involving the progressive proliferation of abnormal leukocytes found in hemopoietic tissues, other organs and usually in the blood in increased numbers. “Leukemic cells” refers to leukocytes characterized by an increased abnormal proliferation of cells. Leukemic cells may be obtained from a subject diagnosed with leukemia.

The term “acute myeloid leukemia” or “acute myelogenous leukemia” (“AML”) refers to a cancer of the myeloid line of blood cells, characterized by the rapid growth of abnormal white blood cells that accumulate in the bone marrow and interfere with the production of normal blood cells. Pre-leukemic conditions such as myelodysplastic or myeloproliferative syndromes may also develop into AML.

The term “tumor” refers to a collection of cancer cells. In one embodiment, the tumor is a leukemia tumor such as an AML cell. In one embodiment, the tumor is a blood tumor.

The term “subject” as used herein includes all members of the animal kingdom including mammals, and suitably refers to humans. Optionally, the term “subject” includes mammals that have been diagnosed with cancer or are in remission. In one embodiment, the subject has been treated, or is concurrently being, treated with chemotherapy, optionally with cytarabine and/or azacytidine.

In one embodiment, the methods and uses described herein involve the administration or use of an effective amount of enhanced T cells, or an effective amount of T cells and Venetoclax.

As used herein, the phrase “effective amount” or “therapeutically effective amount” means an amount effective, at dosages and for periods of time necessary to achieve the desired result. For example, in the context or treating cancer, an effective amount is an amount that for example induces remission, reduces tumor burden, and/or prevents tumor spread or growth of cancer cells compared to the response obtained without treatment. In one embodiment, an effective amount of Venetoclax is an amount that increases T cell-mediated anti-tumor activity and/or increases T cell-mediated cytotoxicity. In one embodiment, an effective amount of enhanced T cells is an amount sufficient to have cytotoxicity against cancer and/or tumor cells in vitro or in vivo.

Effective amounts may vary according to factors such as the disease state, age, sex and weight of the animal. The amount of a given dosage that will correspond to such an amount will vary depending upon various factors, such as the pharmaceutical formulation, the route of administration, the type of disease or disorder, the identity of the subject or host being treated, and the like, but can nevertheless be routinely determined by one skilled in the art. In one embodiment, the enhanced T cells, or T cells and Venetoclax are administered to a subject by injection. In one embodiment, the injection is an intravenous injection. In one embodiment, the injection is a subcutaneous injection, optionally at the tumor site.

In one embodiment, the enhanced T cells, or the combination of T cells and Venetoclax may be used to reduce the growth or proliferation of cancer cells in vitro, ex vivo or in vivo. As used herein, “reducing the growth or proliferation of a cancer cell” refers to a reduction in the number of cells that arise from a cancer cell as a result of cell growth or cell division and includes cell death. The term “cell death” as used herein includes all forms of killing a cell including cell lysis, necrosis and/or apoptosis. In one embodiment, the enhanced T cells, or the combination of T cells and Venetoclax may be used to kill cancer cells in vitro, ex vivo or in vivo.

In one embodiment, the enhanced T cells, or T cells and/or Venetoclax may be formulated for use or prepared for administration to a subject using pharmaceutically acceptable formulations known in the art. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The term “pharmaceutically acceptable” means compatible with the treatment of animals, in particular, humans.

In one embodiment, T cells and Venetoclax are administered to the subject at the same time, optionally as a composition comprising the T cells and Venetoclax, or as two separate doses. In one embodiment, the T cells and Venetoclax are used or administered to the subject at different times. For example, in one embodiment, the T cells are for use or administered prior to, or after administering Venetoclax. In one embodiment, the T cells are for use or administered prior to, or after Venetoclax separated by a time of less than about 1 minute, 2 minutes, 5 minutes, 10 minutes, 30 minutes, 45 minutes, 1 hour, 1.5 hours, 2 hours, 3 hours, 4 hours, 5 hours, 8 hours, 10 hours, 12 hours 16 hours, or 24 hours. In one embodiment, the T cells are for use or administered prior to, or after Venetoclax separated by a time of less than about 1 day, 2 days, 3 days, 4 days, 5 days, 6 days or 7 days.

In one embodiment, Venetoclax is for use or administration to achieve a concentration in the subject of least 25 nM, 50 nM or 100 nM. In one embodiment, Venetoclax is for use or administration to achieve a concentration in the subject of at least 100 nM, at least 200 nM, at least 300 nM or at least 400 nM. In one embodiment, the concentration of Venetoclax of least 25 nM, 50 nM, 200 nM, 300 nM or 400 nM is established concurrently with the administration or use of exogenous T cells, optionally DNTs.

In one embodiment, Venetoclax is for use or administration at a daily dose of between 50 mg and 800 mg, optionally between 100 mg and 600 mg. For example, in one embodiment Venetoclax is for use or administration to the subject in combination with the use or administration of T cells such that the T cells are enhanced by Venetoclax in vivo.

The following non-limiting examples are illustrative of the present disclosure:

### EXAMPLE 1: VENETOCLAX INCREASES THE POTENCY OF T CELL MEDIATED CYTOTOXICITY

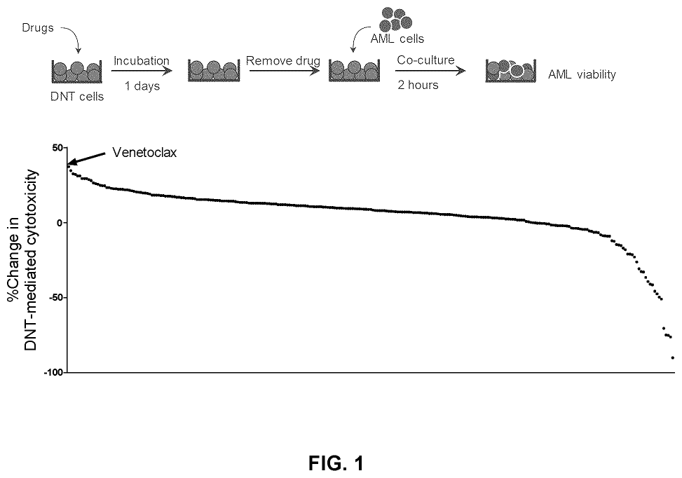

To identify molecules that increase the potency of T cell mediated cytotoxicity against AML, ex vivo expanded DNT cells were used as a surrogate for anti-leukemic T cells and pretreated with a compound library of 269 drugs approved for various clinical uses. Subsequently, compound-treated cells were used as effectors against human AML cell line, OCI-AML2. The Bcl-2 inhibitor, Ven, increased the cytotoxicity of DNT cells the most (FIG. 1).

Ven has largely been used to treat chronic lymphocytic leukemia (CLL) and small lymphocytic leukemia, where Ven inhibits activity of the anti-apoptotic molecule, Bcl-2, promoting apoptosis of malignant cells. Ven as monotherapy has an overall response rate of 64.8%-79.4% for relapse/refractory CLL patients (9). More recently, Ven has been FDA approved to be used alongside with a hypomethylating drug, azacytidine or decitabine, for AML patient treatment, as these drugs significantly improved outcomes of treatment-naïve AML patients that are unfit for other conventional treatments (18, 20), though, the underlying mechanisms are not well understood. Further, immune-stimulatory activities of Ven has not been previously reported.

### EXAMPLE 2: PRETREATMENT WITH VEN INCREASES CYTOTOXICITY OF DNT CELLS AGAINST THREE DIFFERENT AML CELL LINES IN A DOSE-DEPENDENT MANNER

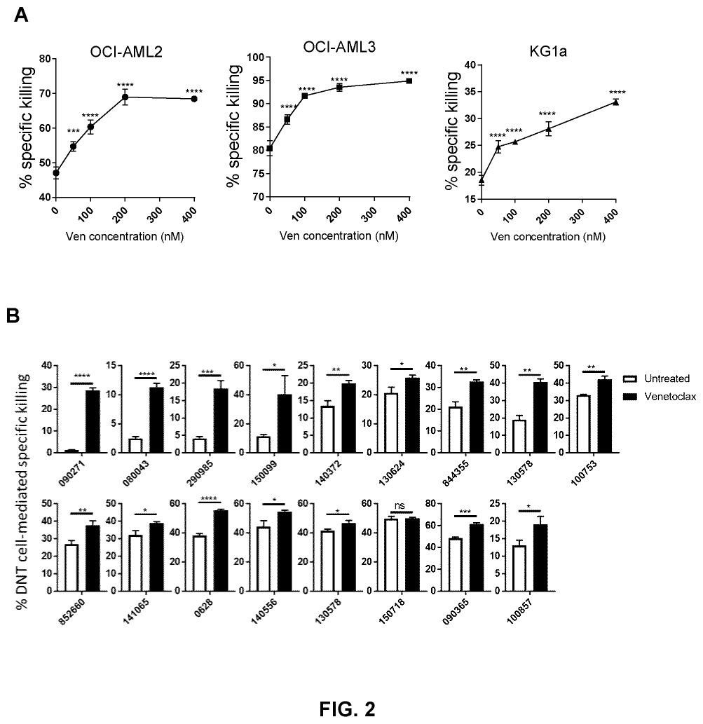

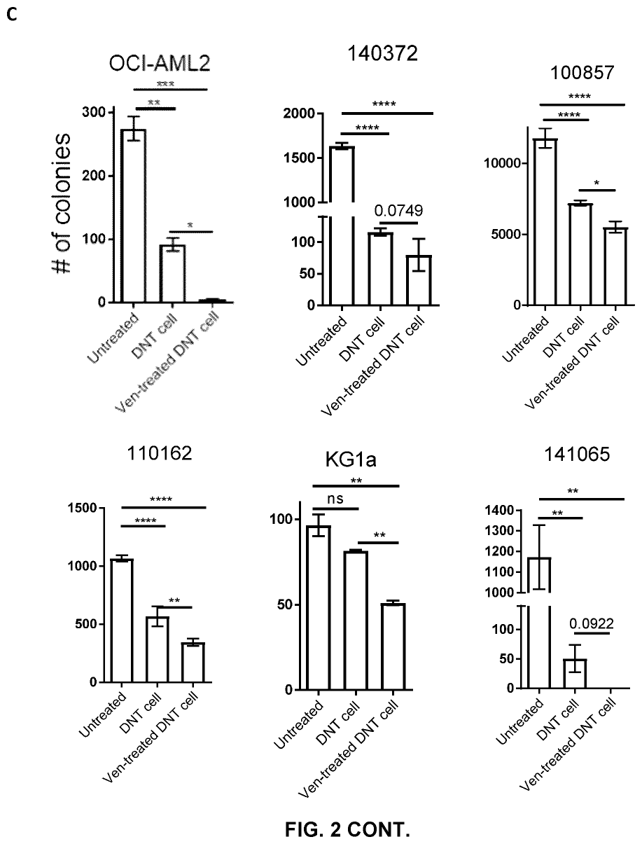

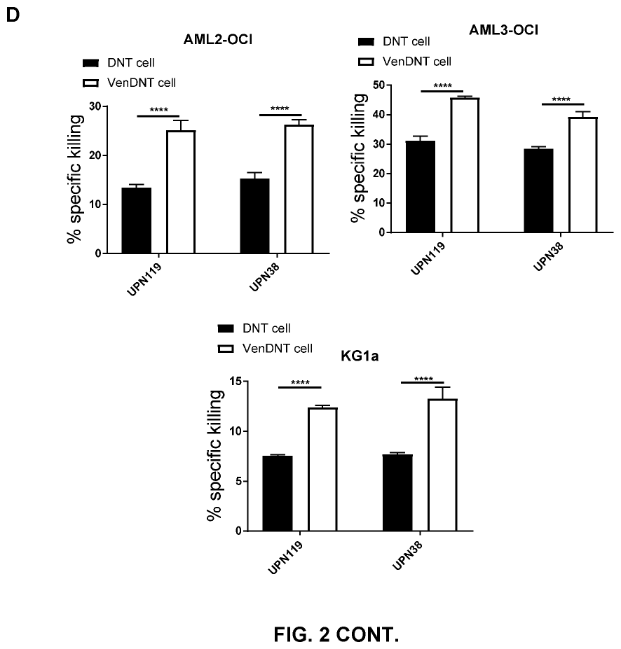

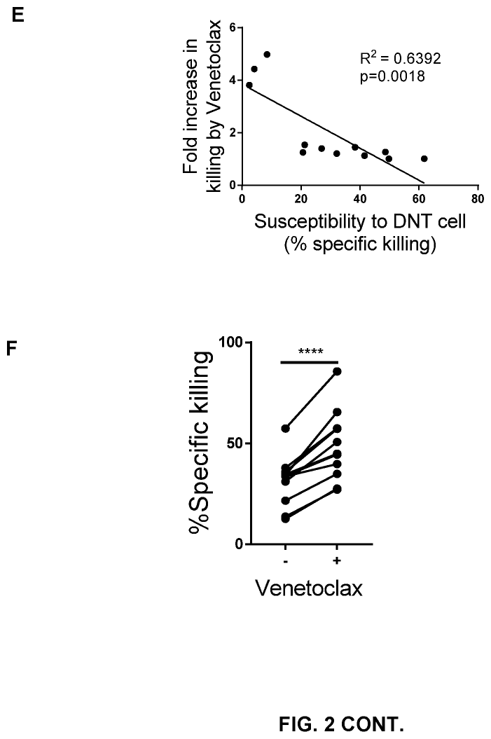

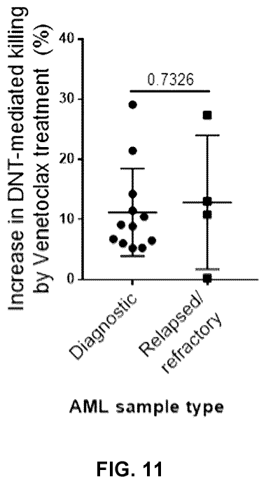

To validate the finding from the drug screening, DNT cells were pretreated with various concentrations of Ven. Pre-treatment with Ven increased cytotoxicity of DNT cells against three different AML cell lines, AML2-OCI, AML3-OCI, and KG1a, in a dose-dependent manner (FIG. 2A). Ven-treated DNTs also showed superior cytotoxicity against 16 out of 17 primary AML samples compared to untreated DNTs (FIG. 2B) Notably, four samples (090271, 080043, 290985, and 150099) that were resistant to DNTs were effectively killed by Ven-treated DNTs. Ven-treated DNTs were equally effective at killing AML cells from patients at diagnosis and relapsed/refractory after induction chemotherapy (FIG. 11). Further, Venetoclax-treated DNT cells also more effectively reduced the colony formation of AML cell lines, AML2-OCI and KG1a, and primary AML blasts, demonstrating an effect on leukemia initiating cells (FIG. 2C) (9-12).

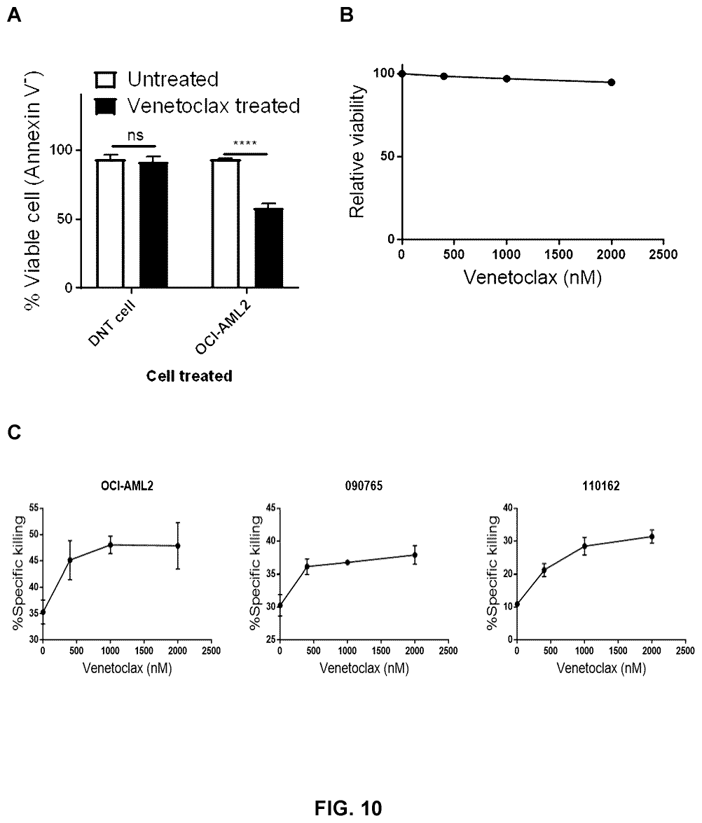

As shown in FIG. 2A, DNT cells pre-treated with varying concentrations of Ven showed a dose-dependent increase in cytotoxicity against three AML cell lines, OCI-AML2, OCI-AML3, and KG1a. This increased effector activity by Venetoclax treatment was retained for at least four days after the removal of the drug from the DNT cells (FIG. 2D). A clear inverse correlation between the susceptibility of AML to DNT cells and the degree of increase in DNT cell-mediated cytotoxicity by Ven treatment was observed (FIG. 2E). Venetoclax increased the DNT-mediated killing of AML cells (FIG. 2A) without reducing the viability of the DNTs (FIG. 10). Increased anti-leukemic activity in Venetoclax-treated DNTs (Ven-treated DNTs) was seen in DNTs derived from all eleven tested DNT donors with an average increase of 61.25% ±31% (FIG. 2F).

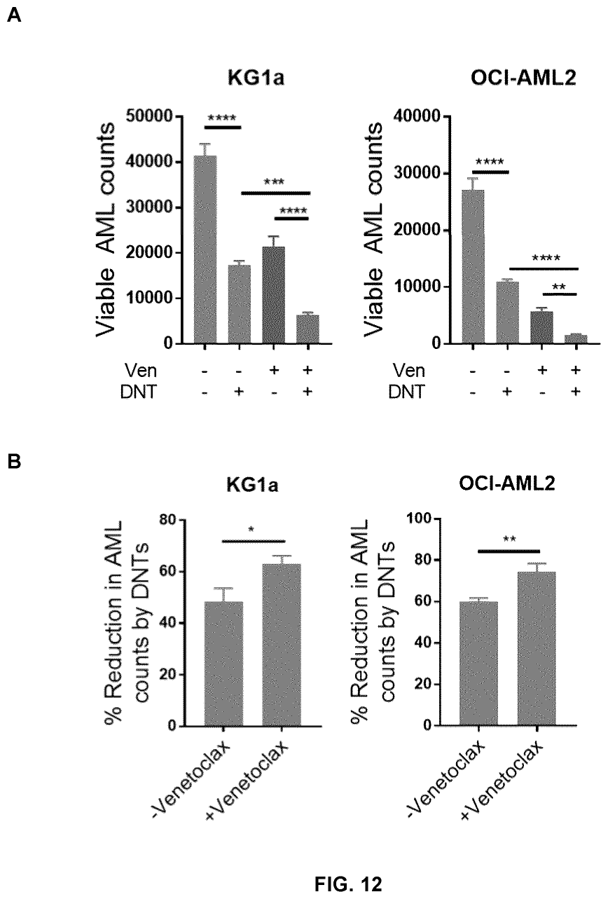

To determine the anti-leukemic activity of DNTs in the presence of Venetoclax, KG1a and OCI-AML2 were treated with Venetoclax, DNTs, or both. Treating AML cells with both DNTs and Venetoclax resulted in a lower number of viable AML cells than either treatment alone (FIG. 12).

### EXAMPLE 3: VEN-TREATED DNT CELLS INDUCED A SIGNIFICANTLY GREATER REDUCTION IN BOTH TUMOR VOLUME AND TUMOR WEIGHT THAN UNTREATED DNT CELLS

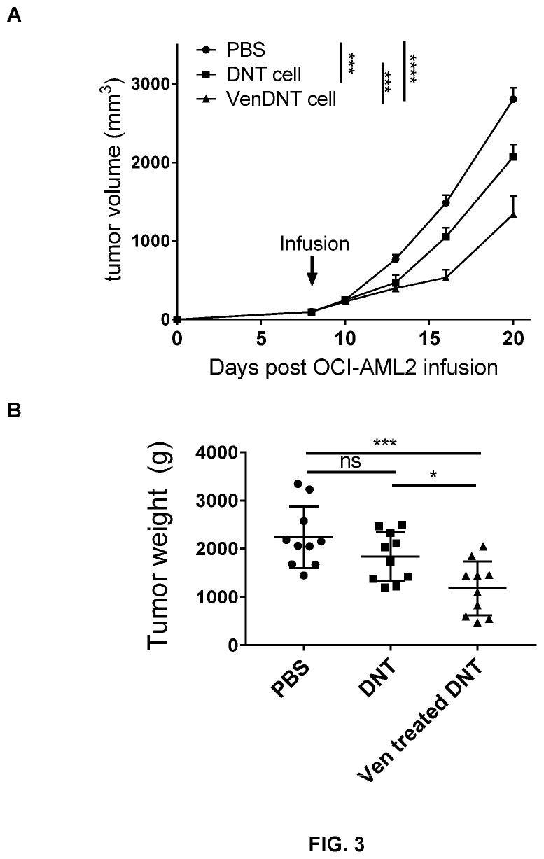

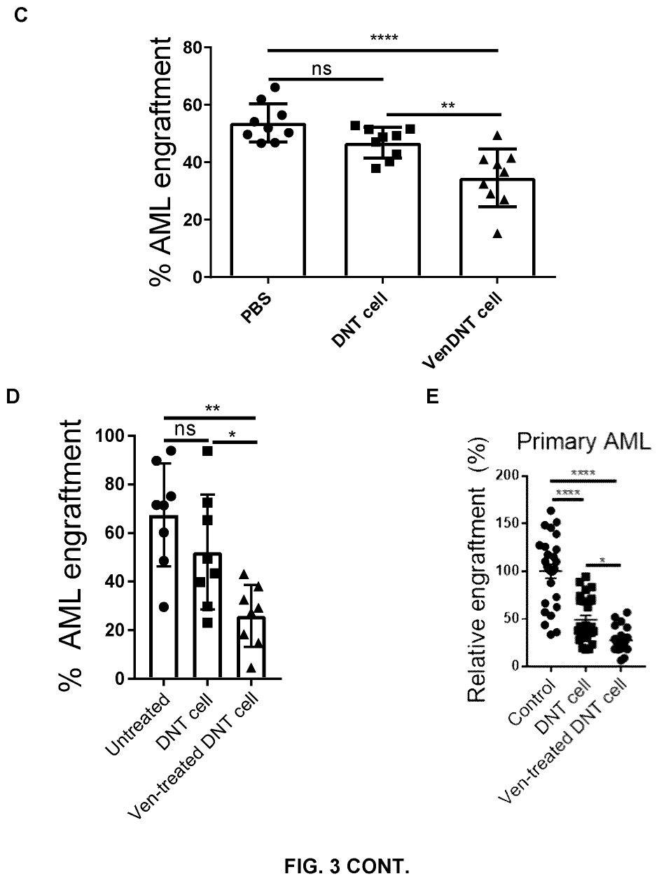

Next, whether ex vivo treatment of expanded T cells with Ven increases their therapeutic efficacy was investigated in vivo using a xenograft model. Immunodeficient mice were subcutaneously inoculated with human leukemic cells. After the tumors were established (>100 mm³ in size), these mice were intravenously infused with a single dose of either non-drug treated or Ven-treated DNT cells, and tumor growth was monitored. While DNT cell treatment effectively targeted leukemia as reported previously (4-6), treatment with VenDNT cells further reduced the tumor volume (26.15% ±5.724% for DNT and 52.23% ±8.468% for VenDNT treated groups on day 20, respectively; FIG. 3A). Similarly, the tumor weights were significantly lower in mice treated with VenDNT cells than those treated with PBS or DNT cells (FIG. 3B). These data demonstrate that Ven treated DNT cells can more effectively target AML cells in vivo. Given that AML primarily resides in the bone marrow (BM), next, whether VenDNT cells can more effectively target bone marrow engrafted AML was studied. Previous reports showed the highly resistant nature of KG1a to DNT cell treatment in a xenograft model (6). Although DNT cells had minimal effect, VenDNT cell treated mice show significantly lower levels of KG1a engraftment compared to PBS and DNT cell treated groups, further supporting the superior anti-leukemic activity of VenDNT cells even against those otherwise resistant (FIG. 3C).

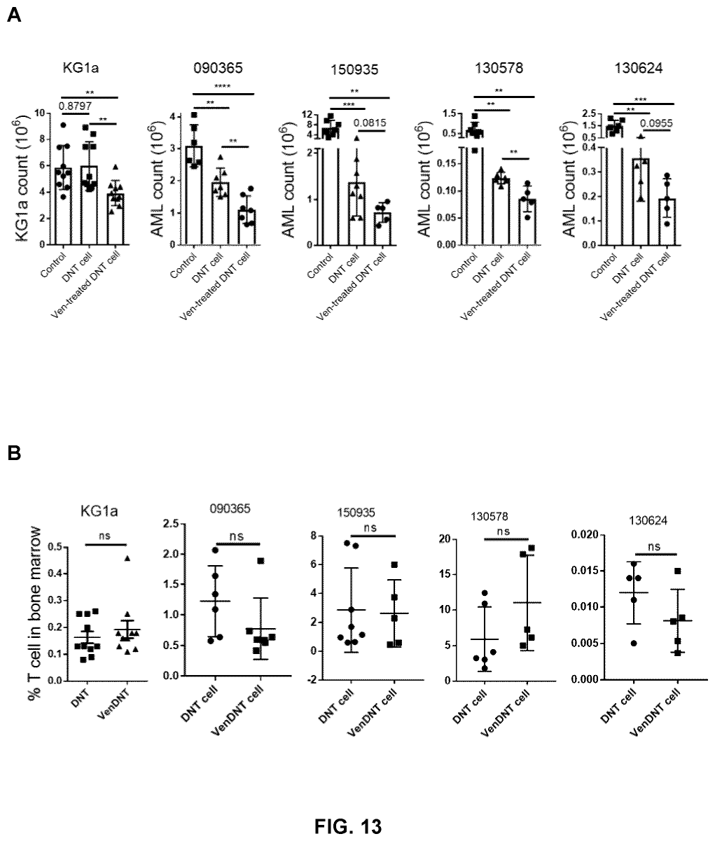

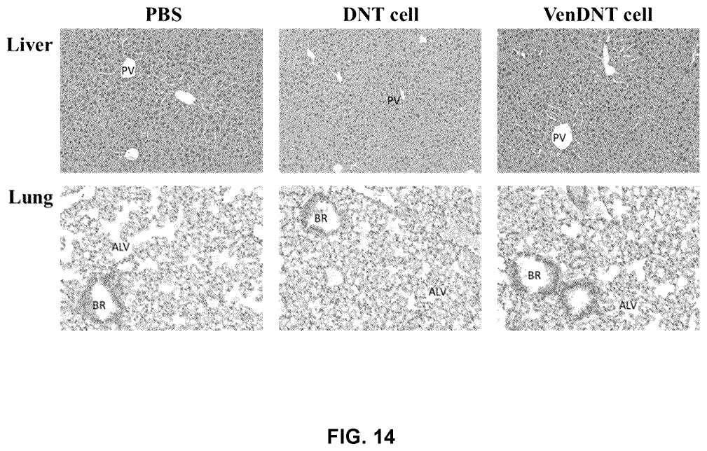

A primary AML sample treated ex vivo with Ven-treated DNT engrafted less than the same cells treated with DNTs alone (FIG. 3D). The effects of Ven-treated DNTs on the engraftment of primary AML samples were further examined. Mice were injected intravenously with primary AML cells and then treated with DNTs or Ven-treated DNTs. Treatment of mice with Ven-treated DNTs decreased AML engraftment and counts compared to mice treated with vehicle control or DNTs (FIG. 3E and FIG. 13A). Similar frequencies of T cells were detected in DNT and Ven-treated DNT groups (FIG. 13B), suggesting that superior anti-leukemic activity of Ven-treated DNTs is due to improved function rather than improved persistence or proliferation of DNTs. Importantly, no notable toxicity was observed from these treatments (FIG. 14).

### EXAMPLE 4: VEN INCREASES THE CYTOTOXICITY OF CONVENTIONAL T CELLS

While treating DNT cells with Ven to enhance their anti-leukemic activity may be beneficial for DNT-therapy, experiments were performed to determine whether Ven has an effect on the anti-leukemic activity of CD4⁺ or CD8⁺ conventional T (T_{conv}) cells as T_{conv} cells are more wildly used as cancer immunotherapy clinically. To this end, polyclonally activated T_{conv} cells were pre-treated with different concentrations of Ven prior to their co-culture with

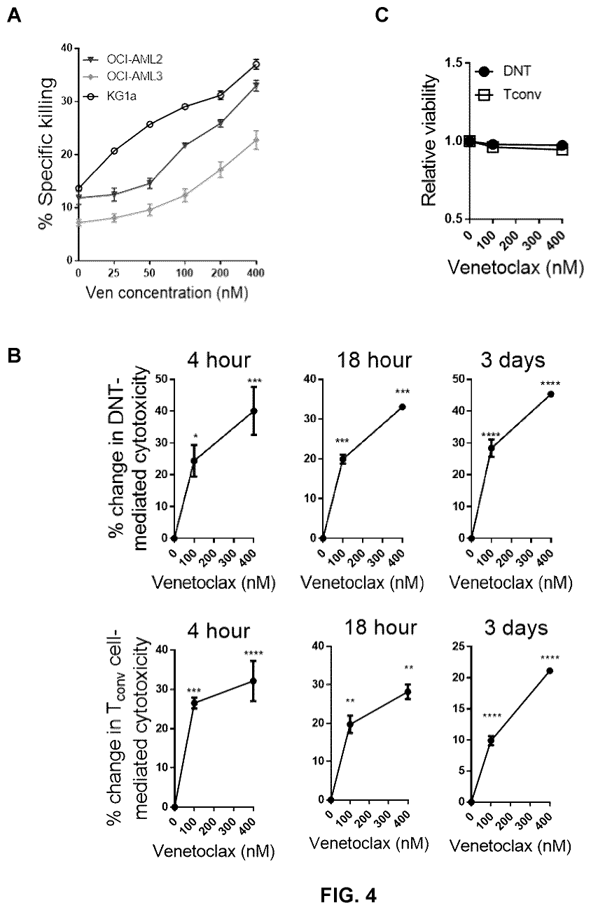

AML cell lines. Similar to what was seen in DNT cells, a significant enhancement of cytotoxicity of T_{conv} cells against various AML cell lines was observed (FIG. 4). These data indicate that Ven can increase anti-leukemic activity of both T_{conv} and DNT cells and support the use of Ven in combination with adoptive T cell therapy to further enhance treatment efficacy.

### EXAMPLE 5: VEN UNIQUELY INCREASES DNT CYTOTOXICITY COMPARED TO OTHER BCL-2 INHIBITORS

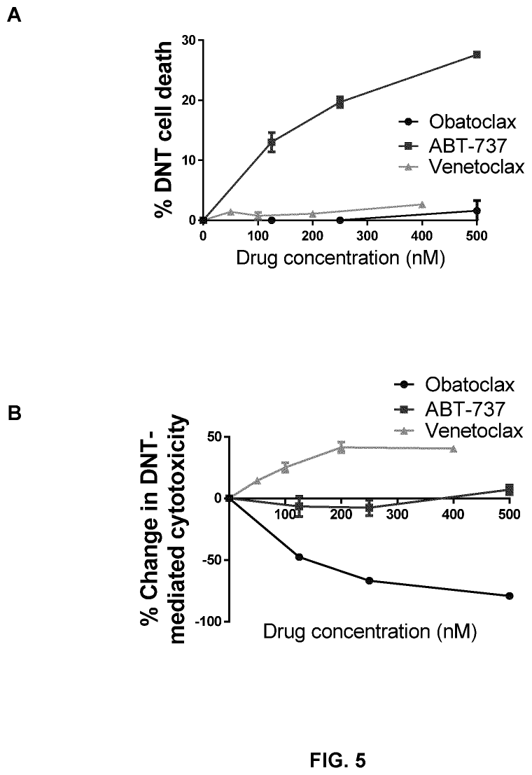

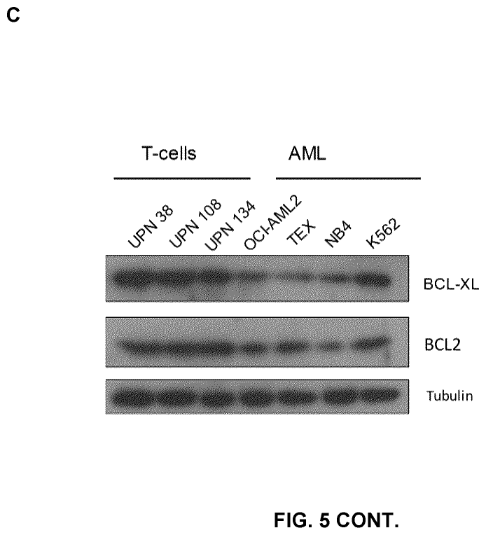

As Bcl-2 is well-known to protect cells from apoptosis, one would expect that inhibiting this pathway would lead to increased T cell apoptosis and dampened T cell function. Given this unexpected finding that Ven increased T cell mediated cytotoxicity, it was next determined whether inhibition of other anti-apoptotic Bcl-2 family proteins have a similar effect using a pan-inhibitor of Bcl-2 family protein, Obatoclax, and a Bcl-2, Bcl-xL, and, Bcl-w inhibitor, ABT-737. In contrast to Ven, these Bcl-2 family protein inhibitors induced DNT cell death or inhibited their cytotoxicity (FIGS. 5A and 5B). Relatively higher expressions levels of Bcl-xL on DNT cells than on AML cells (FIG. 5C) suggests that DNT cells may develop resistance to Bcl-2 inhibition through compensatory activities of Bcl-xL.

### EXAMPLE 6: VEN TREATMENT INCREASES DNT EFFECTOR MOLECULE AND ACTIVATION MARKER EXPRESSION AND ROS LEVELS

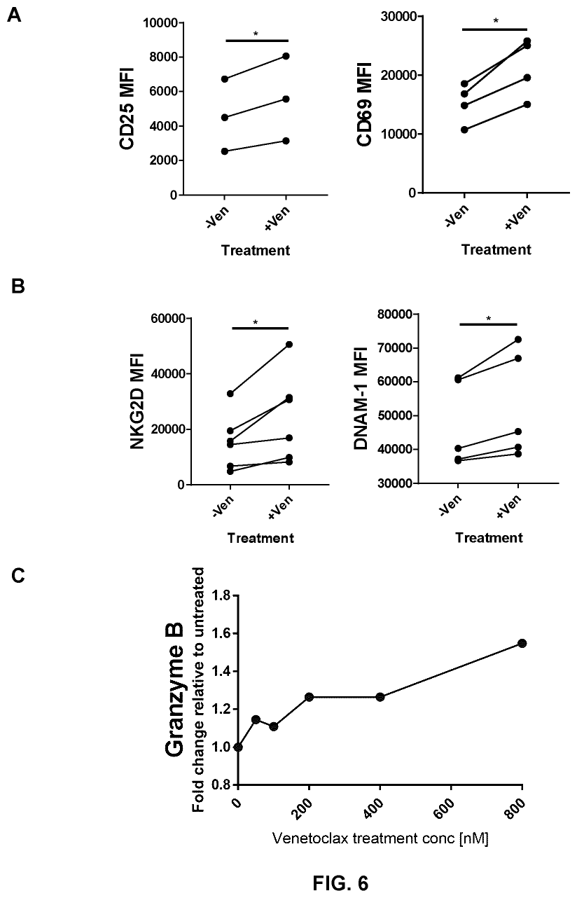

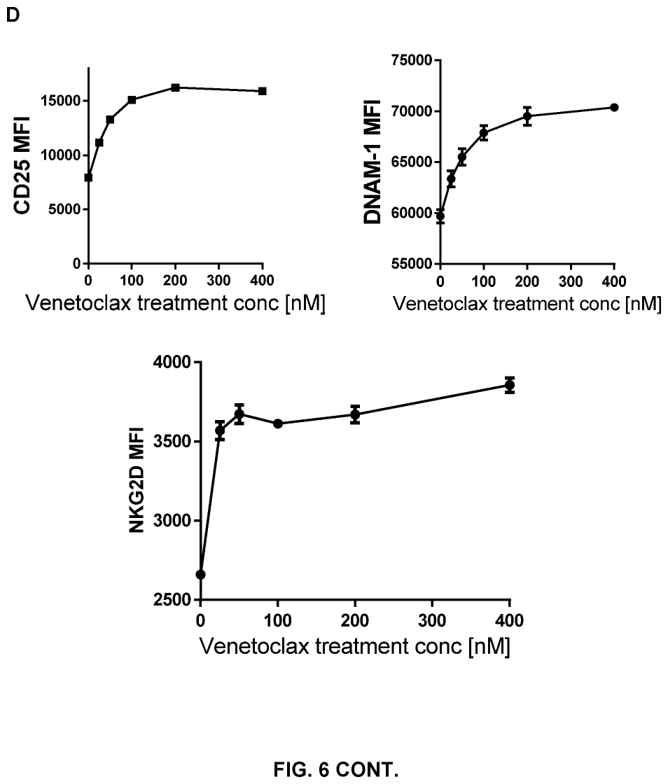

To elucidate the underlying mechanism by which Ven-mediates increased cytotoxicity of T cells, the expression of T cell activation markers and effector molecules on DNT cells with or without Ven treatment was compared. Ven treatment resulted in higher expression of activation markers, CD69 and CD25 (FIG. 6A) and effector molecules NKG2D and DNAM-1 on DNT cells (FIG. 6B). DNT cells treated with Ven also expressed higher levels of granzyme B than vehicle treated ones (FIG. 6C). Similarly, a dose-dependent increase in CD25, NKG2D, and DNAM-1 expression was also observed on Ven treated CD8⁺ (FIG. 6D) T_{conv} cells. Treatment of DNTs and T_{conv} cells with Venetoclax for as little as 4 hours and up to 3 days increased T cell cytotoxicity against AML (FIG. 4B) with increased expression of T cell activation markers (CD69 and CD25, FIG. 6A) and activating receptors (NKG2D and DNAM-1;FIG. 6B) without changing the T cell viability (FIG. 4C). Thus, Venetoclax directly activates effector T cells to increase their cytotoxicity without depleting naïve or inhibitory T cell subsets.

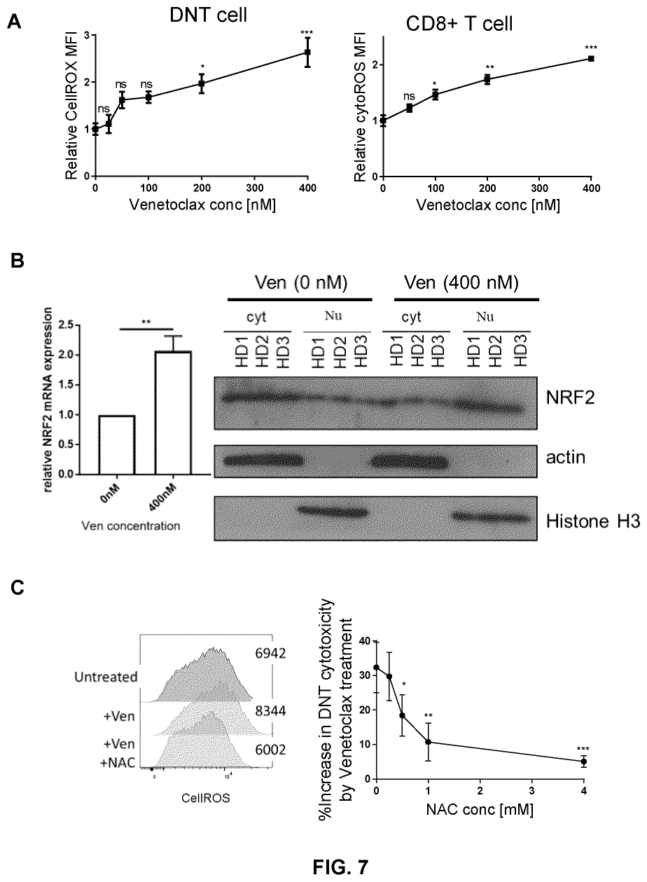

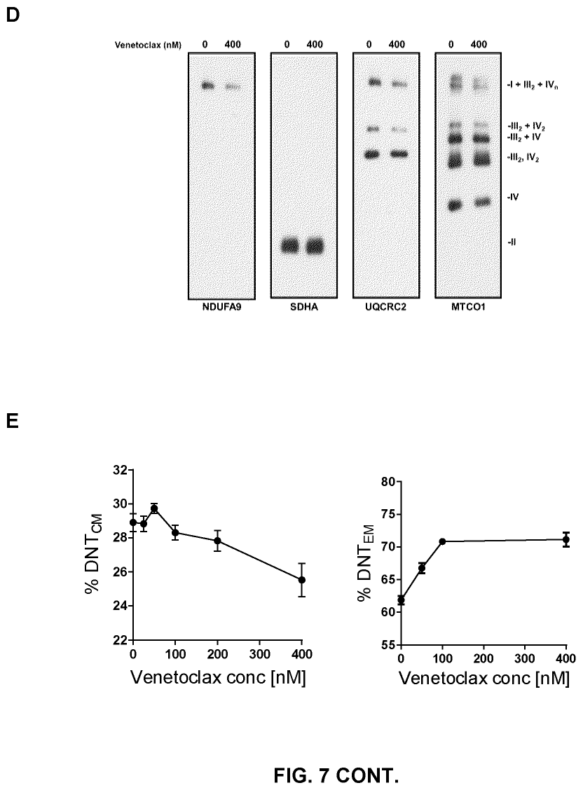

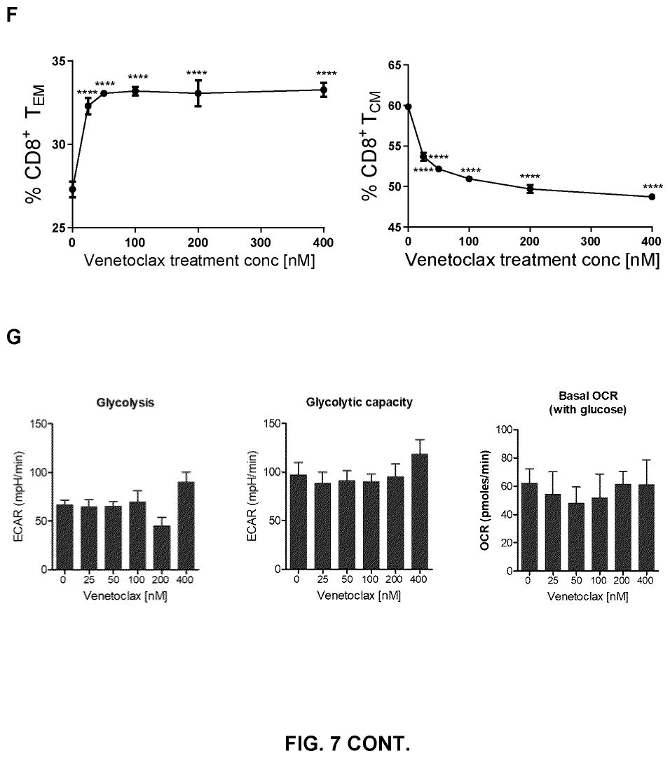

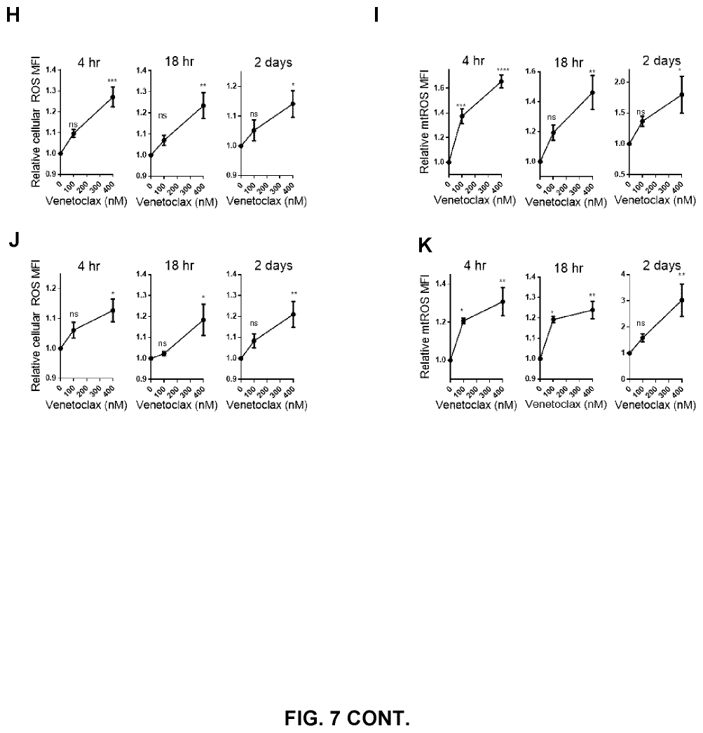

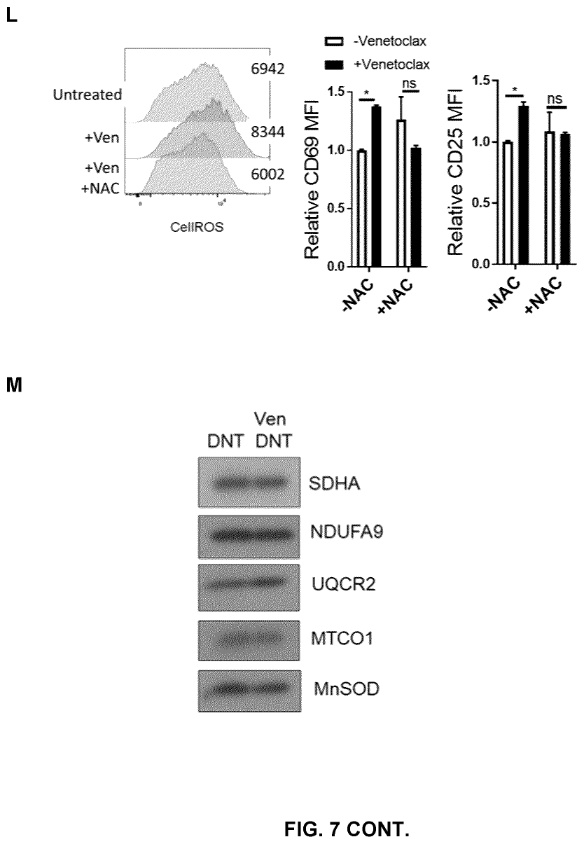

A recent study reported that Venetoclax increases ROS level, and ROS plays an important role in the T cell activation signaling cascade (9, 13-15). However, whether Ven increases ROS level in T cells and augments T cells activation have not been previously reported. To determine the involvement of ROS in Venetoclax-mediated T cell activation and potentiation, cellular and mitochondrial ROS levels in DNT cells and CD8+ T_{conv} cells treated with increasing concentrations of Venetoclax were measured. Venetoclax increased cell cellular ROS in DNT and CD8+ T_{conv} cells in a dose-dependent manner (FIG. 7A). Increased ROS levels were observed despite a compensatory increased expression and nuclear localization of the antioxidant Nrf2 (FIG. 7B).

To determine the functional relevance of higher ROS levels in the Venetoclax-treated DNT cells, we co-treated DNT cells with Venetoclax and increasing concentrations of N-acetylcysteine (NAC). Treatment with Venetoclax and NAC reduced the cellular ROS level and abrogated the effect of Venetoclax on DNT cell-mediated cytotoxicity against AML (FIG. 7C), thus demonstrating the functional relevance of elevated ROS level in Venetoclax-DNT cells. As shown in FIG. 7L, DNTs co-treated with Venetoclax and increasing concentrations of N-acetylcysteine (NAC), a ROS scavenger. abrogated Venetoclax-induced ROS generation and blocked the upregulation of activation markers.

Venetoclax increases ROS generation in malignant cells (9, 21), and ROS plays an important role in the T cell activation and differentiation (15, 22-24). To further understand the mechanism by which Venetoclax activates T cells, we measured ROS generation in DNT and T_{conv} cells treated with Venetoclax. Venetoclax increased cellular and mitochondrial ROS in DNT and T_{conv} cells at concentrations and times associated with increased T cell effector function (FIGS. 7H-K).

To understand the mechanism by which Ven increases mitochondrial ROS in DNTs, we measured levels of respiratory chain proteins. We observed no change in electron transport chain (ETC) complex I, II and IV, subunits of complexes NDUFA9, UQCRC2 and MTCO1, respectively. ROS production is regulated by the respiratory chain supercomplexes, higher order quaternary structures containing respiratory chain complexes I, III, and IV. Reduction in respiratory chain supercomplexes can be associated with higher mitochondrial ROS production (16, 17). As measured by native gels, Venetoclax reduced the formation of respiratory chain supercomplex formation in DNT cells (FIG. 7D).

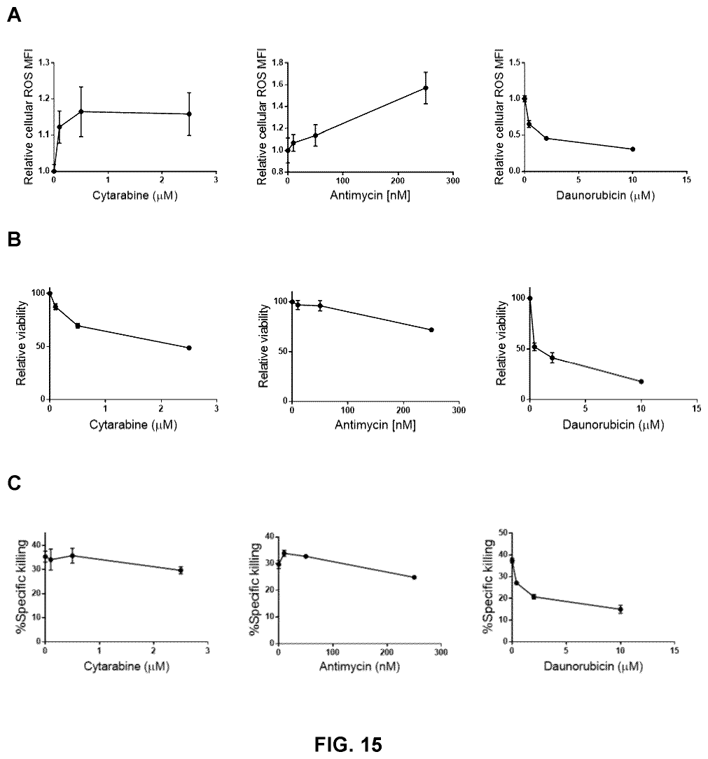

To determine the effect of other ROS-inducing agents on DNT-mediated cytotoxicity, DNTs were treated with increasing concentrations of cytarabine, daunorubicin, and antimycin. Increased ROS levels were observed in DNTs treated with cytarabine and antimycin in a dose-dependent manner with no to little loss of viability (FIGS. 15A and 15B). Daunorubicin treated DNTs had lower ROS level with a large reduction in viability (FIGS. 15A and 15B). Unlike Venetoclax-treatment, cytarabine and antimycin did not enhance the cytotoxicity of DNTs despite the increase in cellular ROS level, and daunorubicin reduced DNT-mediated cytotoxicity against AML (FIG. 15C). These data demonstrate that the ROS-dependent increase in DNT-mediated cytotoxicity is unique for Venetoclax.

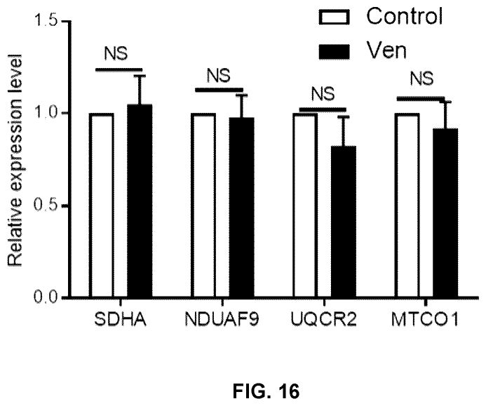

Interestingly, Ven also increased the proportion of cells in effector memory stage while reducing the frequency of central memory T cells for DNT cells (FIG. 7E) and CD8⁺ T_{conv} cells (FIG. 7F). As effector memory T cells preferentially rely on glycolysis while central memory T cells rely on oxidative phosphorylation, and Ven has been shown to inhibit oxidative phosphorylation on AML cells, the level of glycolysis, glycolytic capacity, oxygen consumption rate (OCR) of DNT and VenDNT cells were compared. However, Ven had no significant effect on glycolysis, glycolytic capacity, and basal oxygen consumption rate of DNT cells, suggesting that Ven skews DNT cells towards effector memory phenotype independent of their metabolic pathway (FIG. 7G). To understand the mechanism by which Venetoclax increased ROS production, we measured levels of respiratory chain proteins. No change was observed in NDUFA9, UQCRC2 and MTCO1, subunits of electron transport chain (ETC) complex I, III, and IV, respectively (FIG. 7M and FIG. 16).

Collectively, these results show that Ven activates and skews T cells towards more effector phenotype.

### EXAMPLE 7: VEN-TREATMENT INCREASES THE PROPORTION OF CYTOTOXIC CD8+ AND DNT CELLS IN T CELL POPULATIONS

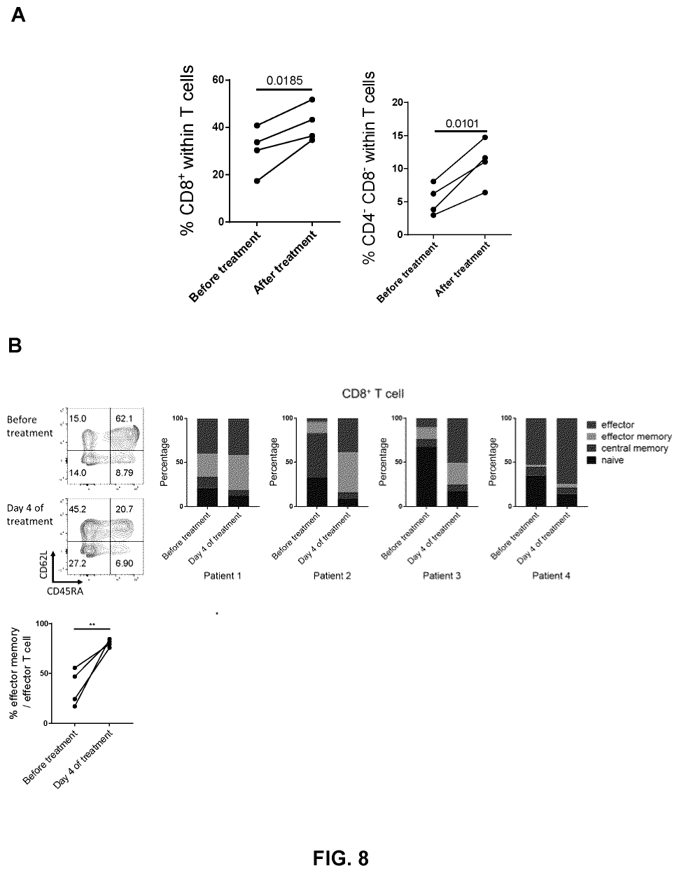

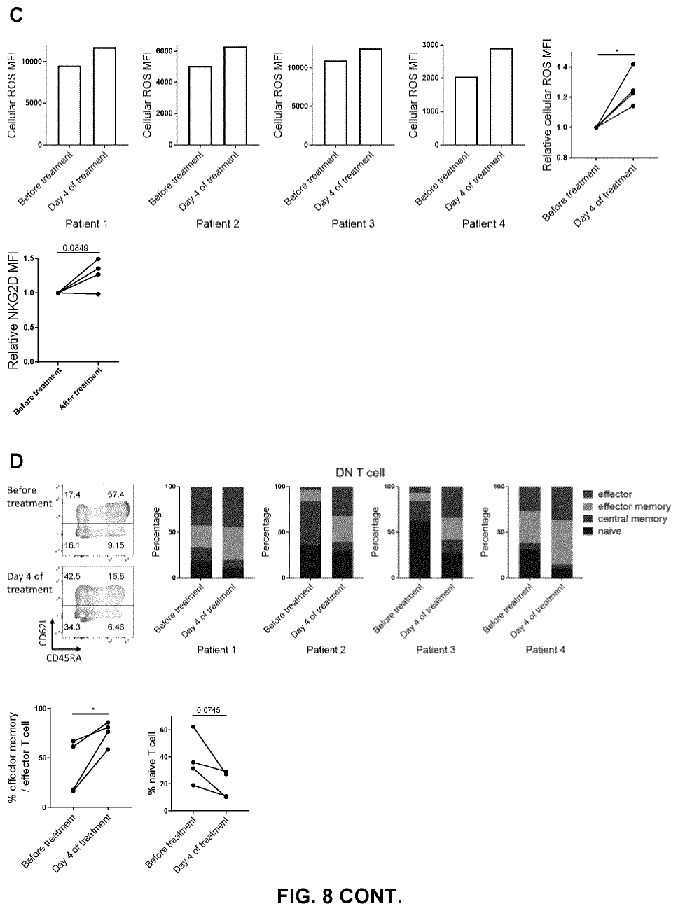

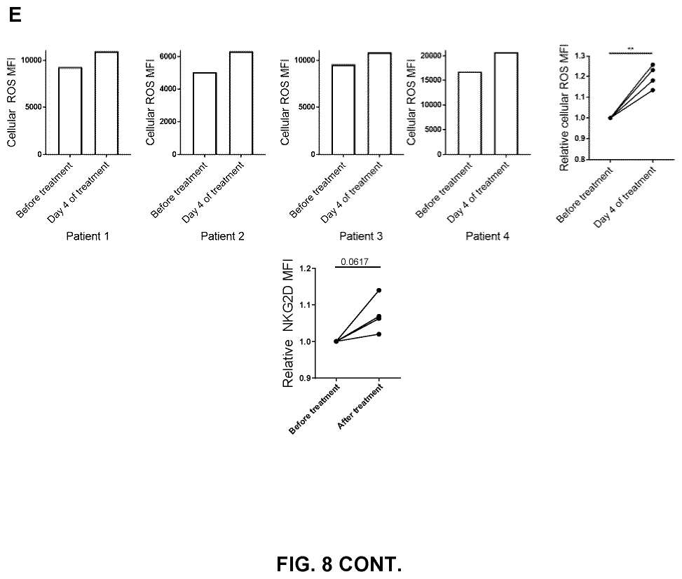

Recent reports indicate Ven and Aza combination therapy given to treatment-naïve AML patients result in significantly improved clinical outcome with low treatment associated toxicities (9 and 19). To determine whether Venetoclax can increase T cell effector activity in patients, we examined T cells from AML patients before and day 4 after treatment with Venetoclax and Azacytidine. Compared to pre-treatment levels, we observed an increase in the proportion of CD8+ and DN T cells after Venetoclax and Azacytidine treatment (FIG. 8A). In agreement with our in vitro findings, increased proportion of CD8+ T cells in effector memory/effector state in all patients (FIG. 8B). Further, NKG2D expression and cellular ROS level on patient CD8+ T cells were increase after treatment (FIG. 8C). Similarly, increased frequency of effector memory/effector subset within DNT cells were seen (FIG. 8D), and, DNT cells also showed higher NKG2D expression and cellular ROS level (FIG. 8E).

### EXAMPLE 8: VEN SELECTIVELY INCREASES THE CYTOTOXIC ACTIVITY OF DNT CELLS AGAINST AML

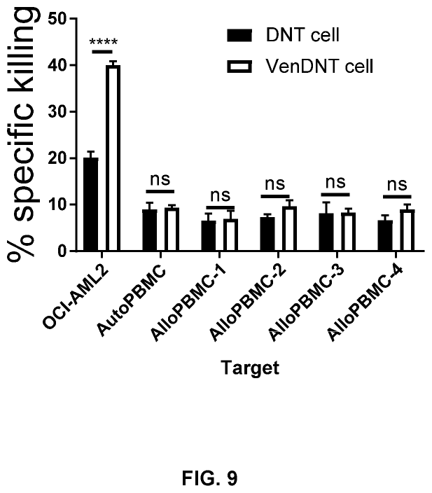

To determine if Ven increases the cytotoxicity of DNT cells against normal blood cells, autologous and allogeneic PBMCs from healthy donors were used as targets. While superior cytotoxicity was seen against OCI-AML2, insignificant degree of killing was seen with both Ven-treated and -untreated DNT cells against autologous and allogeneic PBMCs (FIG. 9), demonstrating that Ven selectively increases the cytotoxic activity of DNT cells against AML.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

1. Park J H, Riviere I, Gonen M, Wang X, Senechal B, Curran K J, et al. Long-Term Follow-up of CD19 CAR Therapy in Acute Lymphoblastic Leukemia. N Engl J Med. 2018;378(5):449-59.

2. Rosenberg S A, Restifo N P. Adoptive cell transfer as personalized immunotherapy for human cancer. Science. 2015;348(6230):62-8.

3. Lee J B, Chen B, Vasic D, Law A D, Zhang L. Cellular immunotherapy for acute myeloid leukemia: How specific should it be? Blood Rev. 2019;35:18-31.

4. Lee J B, Kang H, Fang L, D'Souza C, Adeyi O, Zhang L. Developing Allogeneic Double-Negative T Cells as a Novel Off-the-Shelf Adoptive Cellular Therapy for Cancer. Clin Cancer Res. 2019.

5. Lee J, Minden M D, Chen W C, Streck E, Chen B, Kang H, et al. Allogeneic Human Double Negative T Cells as a Novel Immunotherapy for Acute Myeloid Leukemia and Its Underlying Mechanisms. Clin Cancer Res. 2018;24(2):370-82.

6. Chen B, Lee J B, Kang H, Minden M D, Zhang L. Targeting chemotherapy-resistant leukemia by combining DNT cellular therapy with conventional chemotherapy. J Exp Clin Cancer Res. 2018;37(1):88.

7. Li Q, Cheng L, Shen K, Jin H, Li H, Cheng Y, et al. Efficacy and Safety of Bcl-2 Inhibitor Venetoclax in Hematological Malignancy: A Systematic Review and Meta-Analysis of Clinical Trials. Front Pharmacol. 2019;10:697.

8. Pollyea D A, Stevens B M, Jones C L, Winters A, Pei S, Minhajuddin M, et al. Venetoclax with azacitidine disrupts energy metabolism and targets leukemia stem cells in patients with acute myeloid leukemia. Nat Med. 2018,24(12):1859-66.

9. Jones C L, Stevens B M, D'Alessandro A, Reisz J A, Culp-Hill R, Nemkov T, et al. Inhibition of Amino Acid Metabolism Selectively Targets Human Leukemia Stem Cells. Cancer Cell. 2018;34(5):724-40 e4.

10. Tettamanti S, Marin V, Pizzitola I, Magnani C F, Giordano Attianese G M, Cribioli E, et al. Targeting of acute myeloid leukaemia by cytokine-induced killer cells redirected with a novel CD123-specific chimeric antigen receptor. Br J Haematol. 2013;161(3):389-401.

11. Jin L, Lee E M, Ramshaw H S, Busfield S J, Peoppl A G, Wilkinson L, et al. Monoclonal antibody-mediated targeting of CD123, IL-3 receptor alpha chain, eliminates human acute myeloid leukemic stem cells. Cell Stem Cell. 2009;5(1):31-42.

12. Dick J E. Acute myeloid leukemia stem cells. Ann N Y Acad Sci. 2005;1044:1-5.

13. Chamoto K, Chowdhury P S, Kumar A, Sonomura K, Matsuda F, Fagarasan S, et al. Mitochondrial activation chemicals synergize with surface receptor PD-1 blockade for T cell-dependent antitumor activity. Proc Natl Acad Sci U S A. 2017,114(5):E761-E70.

14. Klein Geltink R I, O'Sullivan D, Pearce E L. Caught in the cROSsfire: GSH Controls T Cell Metabolic Reprogramming. Immunity. 2017;46(4):525-7.

15. Franchina D G, Dostert C, Brenner D. Reactive Oxygen Species: Involvement in T Cell Signaling and Metabolism. Trends Immunol. 2018;39(6):489-502.

16. Lopez-Fabuel I, Le Douce J, Logan A, James A M, Bonvento G, Murphy M P, et al. Complex I assembly into supercomplexes determines differential mitochondrial ROS production in neurons and astrocytes. Proc Natl Acad Sci USA. 2016;113(46):13063-8.

17. Hou T, Zhang R, Jian C, Ding W, Wang Y, Ling S, et al. NDUFAB1 confers cardio-protection by enhancing mitochondrial bioenergetics through coordination of respiratory complex and supercomplex assembly. Cell Res. 2019;29(9):754-66.

18. Roulois, D. et al. DNA-Demethylating Agents Target Colorectal Cancer Cells by Inducing Viral Mimicry by Endogenous Transcripts. Cell 162, 961-973, doi:10.1016/j.cell.2015.07.056 (2015).

19. DiNardo, C. D. et al. Venetoclax combined with decitabine or azacytidine in treatment-naive, elderly patients with acute myeloid leukemia. Blood 133, 7-17, doi:10.1182/blood-2018-08-868752 (2019).

20. Liu, M. et al. Dual Inhibition of DNA and Histone Methyltransferases Increases Viral Mimicry in Ovarian Cancer Cells. Cancer Res. 78, 5754-5766, doi:10.1158/0008-5472.CAN-17-3953 (2018).

21. Nguyen L X T, Troadec E, Kalvala A, et al. The Bcl-2 inhibitor venetoclax inhibits Nrf2 antioxidant pathway activation induced by hypomethylating agents in AML. J Cell Physiol. 2019;234(8):14040-14049.

22. Belikov A V, Schraven B, Simeoni L. T cells and reactive oxygen species. J Biomed Sci. 2015;22:85.

23. Pilipow K, Scamardella E, Puccio S, et al. Antioxidant metabolism regulates CD8+ T memory stem cell formation and antitumor immunity. JCI Insight. 2018;3(18).

24. Mak T W, Grusdat M, Duncan G S, et al. Glutathione Primes T Cell Metabolism for Inflammation. Immunity. 2017;46(4):675-689.

1. A method of enhancing the therapeutic efficacy of T cells, comprising contacting the T cells with Venetoclax to produce enhanced T cells.

2. (canceled)

3. The method of claim 1, comprising contacting the T cells with a concentration of Venetoclax of at least 50 nM, at least 100 nM, at least 200 nM, at least 300 nM or at least 400 nM, optionally a concentration of Venetoclax between about 50 nM and about 1 μM.

4. The method of claim 1, comprising contacting the T cells with Venetoclax for at least about 30 minutes, 1 hour, 2 hours, 4 hours, 6 hours, 8 hours, 12 hours or 24 hours; optionally comprising contacting the T cells with Venetoclax for less than about 14 days, 10 days, 9 days, 8 days, 7 days, 6 days or 5 days.

5. (canceled)

6. The method of claim 1, wherein the T cells are non-conventional T cells, such as double negative (CD4⁻, CD8⁻) T cells (DNTs) or gamma-delta T cells.

7. (canceled)

8. (canceled)

9. The method of claim 1, wherein the T cells are conventional (CD4⁺, CD8⁺) T cells (Tconv).

10. (canceled)

11. (canceled)

12. (canceled)

13. (canceled)

14. The method of claim 1, further comprising removing some or all of the Venetoclax from contact with the enhanced T cells.

15. A population of enhanced T cells produced by the method of claim 1.

16. The population of enhanced T cells of claim 15, wherein the enhanced T cells are non-conventional T cells such as double negative (CD4⁻, CD8⁻) T cells (DNTs).

17. The population of enhanced T cells of claim 15, wherein the enhanced T cells are conventional (CD4⁺, CD8⁺) T cells (T conv).

18. (canceled)

19. (canceled)

20. (canceled)

21. A pharmaceutical composition comprising the enhanced T cells of claim 15 and a pharmaceutically acceptable carrier.

22. (canceled)

23. (canceled)

24. A method of treating cancer in a subject in need thereof, the method comprising administering to the subject the enhanced T cells of claim 15.

25. The method of claim 24, wherein the cancer is leukemia, optionally acute myeloid leukemia.

26. (canceled)

27. A method of reducing the growth or proliferation of a tumor, the method comprising contacting the tumor with the enhanced T cells of claim 15 .

28. (canceled)

29. (canceled)

30. (canceled)

31. (canceled)

32. A method of treating cancer in a subject, the method comprising administering to the subject T cells and Venetoclax.

33. The method of claim 32, wherein the T cells and the Venetoclax are administered to the subject at the same time or at different times, optionally wherein the T cells are administered to the subject within 24 hours, 36 hours or 48 hours of Venetoclax.

34. (canceled)

35. The method of claim 32, wherein the cancer is leukemia, optionally acute myeloid leukemia.

36. A composition comprising T cells and Venetoclax, optionally further comprising a pharmaceutically acceptable carrier or culture media.

37. (canceled)

38. The composition of claim 36, wherein the concentration of Venetoclax in the composition is at least 50 nM, optionally between 50 nM and 1 μM.

39. The composition of claim 36, wherein the T cells are non-conventional T cells such as double negative T cells (DNTs).

40. The composition of claim 36, wherein the T cells are conventional T cells (Tconv).
